(19) United States
(12) Patent Application Publication
(10) Pub. No.: US 2010/0286200 A1
(43) Pub. Date: Nov. 11, 2010

(54) COMBINATION OF METFORMIN AND AN MTP INHIBITOR

(75) Inventors: Ewa Alina Wajs, Beerse (BE); Luc Guillaume M. Van Nueten, Merksplas (BE)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: JANSSEN PHARMACEUTICA NV, Beerse (BE)

(21) Appl. No.: 12/811,917

(22) PCT Filed: Jan. 15, 2009
(86) PCT No.: PCT/EP2009/050418

§ 371 Date: Jul. 7, 2010

(30) Foreign Application Priority Data
Jan. 16, 2008 (EP) 08150296.5

(51) Int. Cl. A61K 31/445; A61P 3/10

(52) U.S. Cl. 514/317

(57) ABSTRACT

The present invention relates to a combination of the anti-diabetic drug metformin and the MTP (microsomal triglyceride transfer protein) inhibitor (+)-phenyl-(4-{4-[(4′-trifluoromethyl-biphenyl-2-carbonyl)-amino]-phenyl}-piperidin-1-yl)-acetic acid methyl ester and the use thereof for combined administration in the treatment or prevention of diabetes mellitus type 2 conditions. The invention further relates to products containing as first active ingredient metformin and as second active ingredient the MTP inhibitor (+)-phenyl-(4-{4-[(4′-trifluoromethyl-biphenyl-2-carbonyl)-amino]-phenyl}-piperidin-1-yl)-acetic acid methyl ester, as combined preparations for simultaneous or sequential use in the treatment or prevention of diabetes mellitus type 2; and to related pharmaceutical compositions and uses.

The present invention relates to a combination of the anti-diabetic drug metformin and the MTP (microsomal triglyceride transfer protein) inhibitor (+)-phenyl-(4-{4-[(4′-trifluoromethyl-biphenyl-2-carbonyl)-amino]-phenyl}-piperidin-1-yl)-acetic acid methyl ester and the use thereof for combined administration in the treatment or prevention of diabetes mellitus type 2 conditions. The invention further relates to products containing as first active ingredient metformin and as second active ingredient the MTP inhibitor (+)-phenyl-(4-{4-[(4′-trifluoromethyl-biphenyl-2-carbonyl)-amino]-phenyl}-piperidin-1-yl)-acetic acid methyl ester, as combined preparations for simultaneous or sequential use in the treatment or prevention of diabetes mellitus type 2; and to related pharmaceutical compositions and uses.

Diabetes mellitus, often simply called diabetes, is a syndrome characterized by a disordered metabolism and inappropriately high blood sugar (hyperglycemia) resulting from either low levels of the hormone insulin produced by the beta cells of the pancreas or from abnormal resistance to the effects of insulin coupled with inadequate levels of insulin secretion to compensate. The World Health Organization recognizes three main forms of diabetes mellitus: type 1, type 2 and gestational diabetes which have different causes and population distributions. Type 1 diabetes is usually due to autoimmune destruction of the pancreatic beta cells. Type 2 diabetes is characterized by insulin resistance or reduced insulin sensitivity in target tissues which causes a need for abnormally high amounts of insulin and diabetes may develop when the beta cells cannot meet demand. Gestational diabetes is similar to type 2 diabetes in that it involves insulin resistance: the hormones of pregnancy can cause insulin resistance in women genetically predisposed to developing this condition.

Type 2 diabetes is by far the most common form of diabetes and most often occurs in overweight or obese adults although the prevalence of the disease in adolescent population dramatically increases. A main component of type 2 diabetes is insulin resistance at the level of the fat and muscle cells. This means the insulin produced by the pancreas cannot connect with cells to let glucose inside and produce energy. This causes hyperglycemia (high blood glucose). To compensate, the pancreas produces more insulin. The cells sense this increase of insulin and become more resistant, resulting in high glucose levels and often times high insulin levels.

When the person with type 2 diabetes cannot achieve normal or near-normal blood glucose levels with diet and exercise, medication may be used to lower blood glucose levels. Type 2 diabetes medications include oral hypoglycemic agents such as metformin, sulphonylureas, thiazolidinediones (rosiglitazone and pioglitazone), meglitinides, sitagliptin, or injectable hormone insulin.

Metformin, or N,N-dimethylimidodicarbonimidic diamide, is the first-line drug of choice for the treatment of type 2 diabetes in overweight and obese people. Metformin therapy improves insulin sensitivity and is not active in the absence of endogenous or exogenous insulin. Its major effect is to decrease hepatic glucose output and thereby lowering fasting glycaemia. Furthermore, metformin enhances peripheral glucose uptake into the muscle and into adipose tissue.

Combinations of metformin with other anti-diabetic agents are widely used in the clinical practice and are subject to intensive clinical investigations. For instance, a combination of metformin with sulphonylurea derivatives such as, e.g. glyburide or glybenclamide, is commonly used as a next step after metformin monotherapy in patients with type 2 diabetes. WO-2005/065663 discloses a combination of rosiglitazone with metformin combination and a combination of pioglitazone with metformin has been recently tested in clinical studies. A combination product of dipeptyl peptidase inhibitor sitagliptin with metformin has been recently approved for the treatment of type 2 diabetes (Janumed). The use of meglitinides with metformin has also been described in US-2007/0167525.

Microsomal triglyceride transfer protein (hereinafter referred as MTP) is known to enable the transport of triglyceride, cholesteryl ester and phospholipids such as phosphatidylcholine predominantly in the intestine and in the liver. This indicates that MTP is required for the synthesis of Apo B-containing lipoproteins such as chylomicrons in the gut and VLDL in the liver. It therefore follows that a systemically active MTP inhibitor would inhibit the synthesis of VLDL and LDL, thereby lowering levels of VLDL, LDL, cholesterol and triglyceride in humans. Compounds capable of inhibiting MTP are believed to be useful in the treatment of disorders such as obesity, hyperlipidemia, hypercholesterolemia, hypertriglyceridemia, type 2 diabetes, atherosclerosis and for the reduction of postprandial serum triglyceride plasma levels.

The MTP inhibitor (+)-phenyl-(4-{4-[(4′-trifluoromethyl-biphenyl-2-carbonyl)-amino]-phenyl}-piperidin-1-yl)-acetic acid methyl ester is described in WO-02/20501 as compound (230). The compound is rapidly metabolised in the plasma, yielding very low systemic plasma concentrations. Therefore it is designed to inhibit predominantly intestinal MTP resulting in the inhibition of triglyceride absorption after the meals but has limited effects on the liver.

It is an object of the invention to provide a combination of the anti-diabetic drug metformin and the MTP inhibitor (+)-phenyl-(4-{4-[(4′-trifluoromethyl-biphenyl-2-carbonyl)-amino]-phenyl}-piperidin-1-yl)-acetic acid methyl ester which has an advantageous effect on the treatment, control or prevention of type 2 diabetes mellitus, in comparison with the mono-therapy treatment of metformin.

This advantageous or beneficial effect can be seen in lower levels of HbA1c (glycosylated hemoglobin) and lower levels of fasting plasma glucose (FPG) when subjects having type 2 diabetes are treated with the combination of said MTP inhibitor and metformin over the mono-therapy treatment with metformin. Other parameters where an advantageous or beneficial effect can be seen are e.g. body weight, LDL levels, and the ratio of LDL over HDL.

HbA1c or glycosylated hemoglobin is a form of hemoglobin that is formed in a non-enzymatic pathway by hemoglobin's normal exposure to high plasma levels of glucose. A buildup of HbA1c within the red cells reflects the average level of glucose to which the cell has been exposed during its life cycle. Measuring HbA1c assesses the effectiveness of anti-diabetic therapy by monitoring long-term glucose regulation. A more effective treatment of diabetes will result in lower levels of HbA1c.

It is believed that the combined use of metformin and the MTP inhibitor (+)-phenyl-(4-{4-[(4′-trifluoromethyl-biphenyl-2-carbonyl)-amino]-phenyl}-piperidin-1-yl)-acetic acid methyl ester stimulates secretion of incretin hormone glucagon-like peptide (GLP-1) which may have beneficial effect on glucose-dependent insulin secretion and also stimulates secretion of peptide YY which induces satiety, thereby producing weight loss and improving insulin sensitivity.

Metformin and the MTP inhibitor (+)-phenyl-(4-{4-[(4′-trifluoromethyl-biphenyl-2-carbonyl)-amino]-phenyl}-piperidin-1-yl)-acetic acid methyl ester may be administered in combination simultaneously (e.g. in separate or unitary compositions) or sequentially in either order. In the latter case, the two active ingredients will be administered within a period and in an amount and manner that is sufficient to ensure that an advantageous effect, or even a synergistic effect is achieved. It will be appreciated that the preferred method and order of administration and the respective dosage amounts and regimes for each active ingredient of the combination will depend on their route of administration and the particular patient being treated. The optimum method and order of administration and the dosage amounts and regime can be readily determined by those skilled in the art using conventional methods and in view of the information set out herein.

As used herein, “administered in combination”, “combined administration” or “combined use” of the MTP inhibitor (+)-phenyl-(4-{4-[(4′-trifluoromethyl-biphenyl-2-carbonyl)-amino]-phenyl}-piperidin-1-yl)-acetic acid methyl ester and the anti-diabetic drug metformin means that these products are administered to a subject at the same time or within an interval such that there is overlap of an effect of each product on the subject.

The present invention also relates to products containing as first active ingredient metformin and as second active ingredient the MTP inhibitor (+)-phenyl-(4-{4-[(4′-trifluoromethyl-biphenyl-2-carbonyl)-amino]-phenyl}-piperidin-1-yl)-acetic acid methyl ester, as combined preparations which are suitable for simultaneous or sequential use in the treatment or prophylactic treatment of patients suffering from diabetes mellitus type 2.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. More particularly, in the present invention directed to combination therapy comprising administration of metformin with the MTP inhibitor (+)-phenyl-(4-{4-[(4′-trifluoromethyl-biphenyl-2-carbonyl)-amino]-phenyl}-piperidin-1-yl)-acetic acid methyl ester, “therapeutically effective amount” shall mean that amount of the combination of agents taken together or sequentially so that the combined effect elicits the desired biological or medicinal response. For example, the therapeutically effective amount of metformin is known to be between 500 mg and 3000 mg per day, usually split in 2 or 3 doses. Extended release formulation tablets may be administered once daily. However in the clinical practice a dose higher than 2000 g/day is given rarely. Further, it will be recognized by one skilled in the art that in the case of co-therapy with a therapeutically effective amount, as in the example above, the amount of the metformin and/or the amount of the MTP inhibitor (+)-phenyl-(4-{4-[(4′-trifluoromethyl-biphenyl-2-carbonyl)-amino]-phenyl}-piperidin-1-yl)-acetic acid methyl ester individually may or may not be therapeutically effective.

It has to be noted that metformin formulation may include an immediate release formulation as well as a sustained or prolonged release (SR) or a novel, extended-release formulation, allowing once daily use.

The product containing as first active ingredient metformin and as second active ingredient the MTP inhibitor (+)-phenyl-(4-{4-[(4′-trifluoromethyl-biphenyl-2-carbonyl)-amino]-phenyl}-piperidin-1-yl)-acetic acid methyl ester, as combined preparations for simultaneous or sequential use in the treatment, control or prevention of diabetes mellitus type 2 may comprise a kit. The kit may comprise a container for containing the separate compositions such as a divided bottle or a divided foil packet, wherein each compartment contains a plurality of dosage forms (e.g. tablets) comprising either metformin or the MTP inhibitor (+)-phenyl-(4-{4-[(4′-trifluoromethyl-biphenyl-2-carbonyl)-amino]-phenyl}-piperidin-1-yl)-acetic acid methyl ester. Alternatively, rather than separating the active ingredient-containing dosage forms, the kit may contain separate compartments each of which contains whole dosage which comprises separate compositions. An example of this type of kit is a blister pack wherein each individual blister contains two tablets, one tablet comprising metformin, the other comprising the MTP inhibitor (+)-phenyl-(4-{4-[(4′-trifluoromethyl-biphenyl-2-carbonyl)-amino]-phenyl}-piperidin-1-yl)-acetic acid methyl ester.

Typically the kit comprises directions for the administration of the separate components. Such instructions would cover situations such as:
i. the dosage form in which the components are administered (e.g. oral)
ii. when the component parts of the product are administered at different dosage intervals, or
iii. when titration of the individual components of the combination is desired by the prescribing physician. The container may have deposited thereon a label that describes the contents therein and any appropriate warnings.

An example of such a kit is a so-called blister pack. Blister packs are well known in the packaging industry and are widely used for the packaging of pharmaceutical unit dosage forms such as tablets, capsules, and the like. Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses between the plastic foil and the sheet. Preferably, the strength of the sheet is such that the tablets or capsules can be removed from the blister pack by manually applying pressure on the recesses thereby an opening is formed in the sheet at the place of the recess. Tablet(s) or capsule(s) can then be removed by means of the opening. It may be desirable to provide a memory aid on the kit, e.g. in the form of numbers next to the tablets or capsules whereby the numbers correspond with the days of the regimen during which the tablets or capsules so specified should be ingested. Another example of such a memory aid is a calendar printed on the card e.g. as follows “First Week, Monday, Tuesday . . . etc Second Week, Monday, Tuesday, . . . ” etc. Other variations of memory aids will be readily apparent. A “daily dose” can be a single tablet or capsule or several pills or capsules to be taken on a given day. Also a daily dose of the first compound can consist of one tablet or capsule while a daily dose of the second compound can consist of several tablets or capsules and vice versa. The memory aid should reflect this.

The present invention is also concerned with pharmaceutical compositions comprising a carrier and as first active ingredient metformin and as second active ingredient the MTP inhibitor (+)-phenyl-(4-{4-[(4′-trifluoromethyl-biphenyl-2-carbonyl)-amino]-phenyl}-piperidin-1-yl)-acetic acid methyl ester, preferably, each in an amount producing a therapeutic effect in patients with diabetes mellitus type 2. The invention also concerns a process for preparing the aforementioned pharmaceutical compositions.

The products of the present invention comprising as a first active ingredient metformin and as a second active ingredient the MTP inhibitor (+)-phenyl-(4-{4-[(4′-trifluoromethyl-biphenyl-2-carbonyl)-amino]-phenyl}-piperidin-1-yl)-acetic acid methyl ester as a combined preparation for simultaneous or sequential use or the pharmaceutical compositions of the present invention comprising the combination of metformin and said MTP inhibitor may also be of use in the treatment of obesity or hyperlipidemia.

Where the invention is said to relate to the use of a product or pharmaceutical composition according to the invention for the manufacture of a medicament for e.g. the treatment of a mammal, it is understood that such use is to be interpreted in certain jurisdictions as a method of treatment of a mammal, comprising administering to a mammal in need of such treatment, an effective amount of a product or pharmaceutical composition according to the invention.

Accordingly the present invention also provides a method of treating or preventing diabetes mellitus type 2 in mammalian subjects, which method comprises the combined administration to the subject of (+)-phenyl-(4-{4-[(4′-trifluoromethyl-biphenyl-2-carbonyl)-amino]-phenyl}-piperidin-1-yl)-acetic acid methyl ester and metformin.

The present invention also relates to the use of (+)-phenyl-(4-{4-[(4′-trifluoromethyl-biphenyl-2-carbonyl)-amino]-phenyl}-piperidin-1-yl)-acetic acid methyl ester for the manufacture of a medicament for combined administration with metformin to improve the efficacy of metformin in the treatment, control or prevention of diabetes mellitus type 2.

To prepare the pharmaceutical compositions for use in accordance with the invention, a therapeutically effective amount of a particular compound, in base or acid addition salt form, as the active ingredient is combined in intimate admixture with a pharmaceutically acceptable carrier, which carrier may take a wide variety of forms depending on the form of preparation desired for administration. These pharmaceutical compositions are desirably in unitary dosage form suitable, for administration orally. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, teaspoonfuls, tablespoonfuls and the like, and segregated multiples thereof.

It may be appropriate to administer the required dose of each component of the combination as two or three sub-doses at appropriate intervals throughout the course of treatment. The starter kit may include a plurality of doses having a constant dose of metformin, such as 250 mg, 500 mg or 100 mg and a variable, increasing dose of MTP inhibitor, for example, 5, 10, 15 mg. The starter kit can provide the agents as separate dosage formulations, or alternatively, the starter kit can provide single dosage formulations of two agents. In embodiments where the starter kit includes combined single unit dosages, exemplary unit dosages are as follows (provided in ratios of metformin: MTP inhibitor as mg dosages): 500:5, 500:10; 500:15, 500:20, 500:25; 500:30, 1000:5, 1000:10, 1000:15, 1000:20, 1000:25, 1000:30, 1500:5, 1500:10, 1500:15, 1500:20, 1500:30, 2000:5. 2000:10, 2000:15, 2000:20, 2000:25, 2000:30.

Proof-of-concept study of combinations of metformin and the MTP inhibitor (+)-phenyl-(4-{4-[(4′-trifluoromethyl-biphenyl-2-carbonyl)-amino]-phenyl}-piperidin-1-yl)-acetic acid methyl ester

Safety and efficacy of the MTP inhibitor (+)-phenyl-(4-{4-[(4′-trifluoromethyl-biphenyl-2-carbonyl)-amino]-phenyl}-piperidin-1-yl)-acetic acid methyl ester in the treatment of subjects with type 2 diabetes mellitus undergoing metformin treatment has been investigated. Three dosing regimens of the MTP inhibitor, 5-10-15 mg bid, were compared to placebo in a double-blind, randomized, parallel-group study design of 12 weeks wherein all subjects were following metformin treatment. All subjects remained on a calorically appropriate, low fat (≦30%) diet, being in accordance with the local practice guidelines for the treatment of type 2 diabetes.

Subjects randomized into the study had to have type 2 diabetes for at least 3 months and remained on a stable metformin monotherapy for at least 2 months. There were 88, 84, 91 and 89 patients randomized to placebo, 5 mg, 10 mg and 15 mg BID. Mean age was 55 years and there were slightly more males than females randomized into the study (males:females—54%:46%).

The mean baseline glycosylated hemoglobin (HbA1c) was 7.8±0.8% (mean±SD) and the mean plasma glucose was 9.1±1.80 mmol/L (mean±SD). The majority (68%) of subjects had HbA1c levels≦8%.

Mean weight at baseline was 94.9 kg and varied between 92.8 kg and 96.7 kg in different treatment groups, while the mean baseline BMI was 32.8 kg/m².

Eighty seven percent of subjects completed the study. The proportion of subjects who withdrew from the study was 13% (11/88), 7% (6/84), 18% (16/91) and 13% (12/89) for placebo, 5, 10 and 15 mg bid dose groups, respectively.

Primary efficacy parameter: the change from baseline at Week 12 of HbA1c levels showed a statistically significant decrease vs. placebo for all active treatment arms using a mixed model analysis. The mean decrease in HbA1c (%) in different treatment arms was −0.27, −0.46, −0.55 and −0.54 for placebo, 5, 10 and 15 mg bid, respectively. The least square mean (from the mixed model) difference from placebo was 0.19, 0.28 and 0.28 for 5, 10 and 15 mg bid doses, respectively.

Key secondary efficacy parameters: the mean body weight loss at Week 12 was 1.88 kg (2%), 1.71 kg (1.0%), 2.80 kg (2.9%) and 3.37 kg (3.6% in the placebo, 5, 10 and 15 mg bid groups, respectively. The mean percentage changes from baseline at week 12 in fasting plasma glucose (FPG) in active treatment arms were +0.7%, −2.3%, −6.1%, and −6.0% for the placebo, 5, 10 and 15 mg bid groups, respectively and corresponded to the modest absolute changes from baseline (increase of 0.06 mmol/L and decreases of 0.37. 0.063 and 0.63 mmol/L in placebo, 5, 10 and 15 mg bid dose groups, respectively). The magnitude of the observed changes in the active treatment arms are in agreement with to the drop in HbA1c.

Secondary efficacy parameters measured included fasting plasma lipids. Beneficial effects were seen in LDL levels and the ratio of LDL over HDL when a combination of the MTP inhibitor (+)-phenyl-(4-{4-[(4′-trifluoromethyl-biphenyl-2-carbonyl)-amino]-phenyl}-piperidin-1-yl)-acetic acid methyl ester at 10 mg bid and 15 mg bid and metformin was administered.

1. A product comprising as first active ingredient metformin and as second active ingredient the MTP inhibitor (+)-phenyl-(4-{4-[(4′-trifluoromethyl-biphenyl-2-carbonyl)-amino]-phenyl}-piperidin-1-yl)-acetic acid methyl ester as a combined preparation for simultaneous or sequential use.

2. (canceled)

3. A method of treating or preventing diabetes mellitus type 2, comprising administering the product of claim 1 to a mammal in need of such treatment.

4. A method of treating obesity, comprising administering the product of claim 1 to a mammal in need of such treatment.

5-6. (canceled)

7. A pharmaceutical composition comprising as first active ingredient metformin and as second active ingredient the MTP inhibitor (+)-phenyl-(4-{4-[(4′-trifluoromethyl-biphenyl-2-carbonyl)-amino]-phenyl}piperidin-1-yl)-acetic acid methyl ester, and a carrier.

8. (canceled)

9. A method of treating or preventing diabetes mellitus type 2, comprising administering the pharmaceutical composition of claim 7 to a mammal in need of such treatment.

10. A method of treating obesity, comprising administering the pharmaceutical composition of claim 7 to a mammal in need of such treatment.

11-12. (canceled)

13. A method of improving the efficacy of metformin for the treatment or prevention of diabetes mellitus type 2, comprising combining metformin and (+)-phenyl-(4-{4-[(4′-trifluoromethyl-biphenyl-2-carbonyl)-amino]-phenyl}piperidin-1-yl)-acetic acid methyl ester in a pharmaceutical composition for administration to a subject in need thereof.
